(19) United States
(12) Patent Application Publication
(10) Pub. No.: US 2009/0198211 A1
(43) Pub. Date: Aug. 6, 2009

(54) Convenience IV kits and methods of use

(75) Inventors: Gale H. Thorne, JR., Bountiful, UT (US); Evan W. Call, Bountiful, UT (US); Gale H. Thorne, Bountiful, UT (US)

Correspondence Address: Gale H. Thorne, 1056 Millcrest Circle, Bountiful, UT, 84010, US

(73) Assignee: IntraVena, LLC, Bountiful, UT (US)

(21) Appl. No.: 12/080,185

(22) Filed: Apr. 1, 2008

Related U.S. Application Data
(63) Continuation-in-part of application No. 12/012,837, filed on Feb. 6, 2008.

(51) Int. Cl. A61M 31/00; A61M 39/00; A61M 39/22

(52) U.S. Cl. 604/506; 604/248; 604/533

(57) ABSTRACT

A convenience kit which provides a basic configuration for use in measuring, filling and dispensing medication and flush solutions to IV sets and patient catheters through needleless connectors while improving safety and efficacy by requiring fewer post-sterilization makes and breaks compared to conventional filling and dispensing methods. Further, the kit improves flush compliance by facilitating dispensing of flush solutions and decreases likelihood of infections by providing for flushing of patient lines and catheter connecting fittings without line breaks. Convenience of operation is provided by a two-syringe assembly which is operable by a single hand for selective dispensing accomplished from either of the two syringes while obstructing flow from the other syringe. The kit comprises a clip for stabilizing the two syringe assembly.

### CONTINUATION-IN-PART

This Application for Patent is a Continuation-in-Part of U.S. patent application Ser. No. 12/012,837 filed Feb. 6, 2008 which is made part of this Application by reference.

### FIELD OF INVENTION

This invention relates generally to medical intravenous administration syringes, specifically including pre-filled flush syringes. It is also particularly related to kits and to methods which employ preassembled parts which are substantially fabricated for the purpose of achieving a significant decrease in need for making and breaking line connections and other product manipulations and for reducing dangerous conditions related to administering hazardous drugs.

### BACKGROUND AND DESCRIPTION OF RELATED ART

During the past decade, a great effort has been made by the medical community to decrease concerning and sometimes tragic effects of accidental needle sticks. A revolution in medical needle products and their use has resulted in significant growth of a relatively new safety needle industry. In addition, use of IV catheters has significantly reduced the number of needle sticks required in contemporary medical practice.

However, increased use of catheters (nearly every hospital patient is currently equipped with an IV catheter shortly after admission) has resulted in a generation of problems and procedures related to catheter safety. Recognition of some catheter use problems has resulted in the following principles, considerations and guidelines:
A basic principle taught in IV therapy is that every IV delivered medication should be flushed. This principle is intended to help prevent incompatible drug mixing and assure delivery of a timely, complete dose. Flushing of an administration port associated with a catheter also decreases likelihood of contamination by residual drops or wetted surfaces on the outside of the port. Recognition of a need to clear a “Y-site” following injection of a dose through a port has led to a widespread practice of drawing in flush from an available saline source (such as a hanging bag which communicates with the receiving catheter); however, as is disclosed hereafter, this practice may be unsatisfactorily performed in some critical situations.
Unfortunately, many nurses forget to flush or assume that a running IV will flush a Y-injection site which leaves small amounts of medication in the Y-site where a potentially incompatible drug may cause a problem. Also, many institutions purport that a high catheter replacement rate in central lines is a direct consequence of a failure to consistently flush lines after each medication injection into the IV line.
A Jul. 5, 2005, PHC4 Research Brief entitled “Hospital-acquired Infections in Pennsylvania” reported that clinician-caused (nosocomial) bloodstream infection rates in Pennsylvania may be as high as 21,458 per year at a treatment cost of $861 million and mortality rate of 25.6% in 2004 alone. Such treatment costs in hospitals extrapolate to a $20.3 billion cost and over 80,000 deaths per year in the United States. Additional studies that cite similar increases in infection rates led to the “100,000 lives Campaign” instigated by the Institute for Healthcare Improvements, Cambridge, Mass., which is intended to save lives that would otherwise be lost due to nosocomial infection rates. Clinicians who work in W therapy are well schooled in knowing that “the more line breaks and line manipulations, the greater the chance for line contamination”. Reducing line breaks and line manipulations, in principle, will reduce line contaminations and patient infections. Please note that a simple connection of a flush syringe after disconnection of a dose administration syringe adds an additional make and break to the process.
A chronic nursing shortage, projected to persist beyond 2012, places nursing time at a premium. Short-staffed healthcare facilities result in busier nurses who may be more prone to medical errors, some of which result in serious consequences for patients. A product which would save nursing time by reducing nursing steps would simplify care-giver procedures and by reducing nursing steps may also reduce clinician errors and overall healthcare costs.
A 2004 NIOSH (National Institute of Occupational Safety and Health) Safety Alert: Preventing Occupational Exposure to Antineoplastics and Other Hazardous Drugs in Healthcare Settings warns healthcare institutions about the need to provide products and procedures to protect clinicians from hazardous drug exposure. Attempts to reduce such drug exposure has resulted in use of expensive protective port attachment devices.

Thus, there exists a severe contemporary need for devices, not currently available commercially, which reduce injection site makes and breaks (see Terms and Definitions Section), reduce nurse time, facilitate ease of flushing and provide a greater degree of safety related to line contamination and subsequent patient infection and care-giver risk to hazardous drug exposure.

### TERMS AND DEFINITIONS

Following is a list of terms and associated definitions provided to improve clarity and understanding of precepts of the instant invention:
break, n: a disconnection of a pair of medical connectors, usually as part of a medical procedure
clip, n: a holder for a pair of syringes for stabilizing the syringes while performing a medical procedure
crib pad, n: a pad which comprises a barrier layer and an absorbent layer and which derives its name from pediatric applications
dead space, n: a volume of inaccessible fluid which is retained within a device after a procedure
extension set, n: any tubing and associated connecting parts which may be used for fluid medication delivery through a catheter; generally, such extension sets have ports (now commonly needleless ports) which provide access for fluid delivery
half-life, n: a period of time during which activity or usefulness declines by half (generally applied to drugs which deteriorate quickly when introduced into a physical system)
fitting, n: a medical connector for fluids
IV set, n: intravenous drug delivery tubing specifically dedicated for use with an associated IV catheter and IV container
kit, n: a group of parts, provided within a single package for a designated medical use
luer fitting, n: a medical connector having a frustoconically shaped connecting geometry which is in common use in medical practice
luer lock fitting, n: a luer fitting having a locking mechanism whereby a male and female connector are securely, but releasibly affixed one to the other
make, n: a connection or re-connection of a pair of medical connectors usually made as a result of a medical procedure
needleless connector, n: a fitting which reliably permits needle free fluid access to an IV set or through a vial adapter to a vial, a fitting having connector interface geometry similar to a conventional syringe
port, n: a site for a medical connector, where through fluid is communicated to a patient line (e.g. a catheter)
pouch, n: a bag or tray
short extension set, n: any tubing and associated connecting parts which may be used to connect a stopcock to a pre-filled syringe
subkit, n: a group of parts provided as a unit within a kit being identifiably separate from other parts of the kit (on its own, a subkit could be considered to be a kit)
TPA, n: one of a set of drugs used for clearing blood-clot occluding catheters; other such drugs include stretokinase, urokinase, etc.
unitized, adj: a plurality of separate parts permanently joined to be handled and used as a single unit
wrap, n: a flexible container which may be a bag or folded shield which is sealed to provide a cover in which enclosed parts are sterilized and protected until opened for use

### BRIEF SUMMARY AND OBJECTS OF THE INVENTION

In brief summary, use of this novel invention generally decreases known problems related to makes and breaks and contamination, enumerated supra, while increasing patient safety when dealing with catheter related injection ports and associated devices. The invention involves providing a kit which is used with other components or parts, generally available at an assembly site, to construct an assembly which can be used to substantially reduce inadvertent risk of contamination of hazardous drugs and of infection due to makes and breaks during drug administration. The kit comprises a fluid switching component and short extension set as the basis for a two syringe (i.e. a dose syringe and a flush syringe) assembly, and other basic parts of the assembly disclosed in detail hereafter, which are used to improve safety and efficacy of drug administration.

Use of this kit potentially resolves a number of issues related to conserving nursing and pharmacist time. The invention is a dedicated convenience kit comprising a pouch or wrap containing parts, which are sterilized therein and preferably unitized and which are assembled with the other readily available parts, at an assembly site, for performing a medical procedure involving providing medication through a catheter. This convenience kit may be used as a subkit for a larger convenience kit generally assembled with additional parts for specific medical procedures.

Generally, the kit package is used in two stages, (1) preparation (usually in pharmacy) and (2) delivery (at site of use). As stated supra, kit components comprise a fluid switching component, a short extension set and other items specifically made available for kit use. The kit generally contains those items which are not readily available at a preparation site. The fluid switching component has attachment sites for access to at least two syringes which become part of the kit apparatus when affixed thereto. The fluid switching component has another attachment site through which filling and dispensing fluids is performed. It should be noted that a simple luer attachment may not be sufficient because, though all luer attachments provide connecting geometry which is necessary for connecting to all luer ports, not all provide the necessary geometry required to reliably connect to a needleless connector.

As retaining purity of contents of both drug and flush syringes is critical in most applications involving the kit, the fluid switching component must inherently keep disparate contents of the drug and flush syringes until fluid from each syringe is dispensed into a receiving connector through the other attachment site. Consideration must also be given to physical characteristics of the other attachment site, itself. As stated supra, it is important that the other attachment site interface reliably with all IV set and injection port configurations. It is particularly important that a reliable connection be made with needleless fittings, in general.

As two syringes (dose and flush) are used in tandem during a sequence of drug and flush dispensing, preferably with the use of but a single hand, it is important that both syringes be presented to a user in a manner which is conducive to single-hand operation. For this purpose, the short extension set is provided to permit orienting the flush syringe parallel relative to the dose syringe. Further, a clip provides opportunity to stabilize the syringes for such use.

At a station where kit components and other items are assembled for use (for example, in pharmacy where a syringe is filled with a prescribed drug.) a drug syringe and a flush syringe are affixed to the kit components. Preferably the station is in a controlled environment (such as in a sterile area and/or under a laminar flow hood) so that kit components may be accessed yet remain contamination free. It is preferred that kit components be provided to a preparer in a “ready to use” format which will not inadvertently come apart.

One of the compelling purposes of convenience kits resulting from this invention is providing an inherently associated flush syringe. As contents of a flush syringe should, in most cases, be kept disparate from a prescribed drug prior to drug delivery, it is important that a secure fluid switching component be used to controllably regulate filling and delivery pathways. For this purpose, it is presently preferred to use a stopcock. Even so, other modes of fluid regulation may be used within the scope of the invention. As an example, “Y” sites with clamps on extensions of tubing therefrom may be used. Also other switching components may be used so long as the above disclosed disparate and fitting compatibility criteria are met.

The critical feature is assurance that the flush and drug fluids are kept disparate until delivery. Stopcocks are commonly used in medical practice; however, a stopcock configuration for at least one convenience kit application (for hazardous drugs such as those used in oncology) is not generally available commercially. Disclosure of such a stopcock is provided in detail hereafter.

Once preparation in pharmacy is complete (e.g. the drug syringe prescription is attached and filled) and a flush syringe is affixed to the kit components, with exit pathways capped and protected, the assembled kit components should be labeled and packaged for transport to the site of use following institutional protocol. At a patient delivery site of use, contents of the package are removed and, with but a single make, connected to a catheter dispensing port whereat, using the stopcock as the switching mechanism, the drug syringe is emptied as prescribed, followed by flush delivery to assure compliance with guidelines for flushing.

Handling two syringes affixed to a stopcock may require a fixture to stabilize one of the syringes while using the other. For such purposes, a dual syringe clip is an element of the instant invention provided to facilitate syringe handling.

One example of a convenience kit assembly, based upon the present invention, is a hazardous drug kit assembly. While nearly all drugs may be considered to be somewhat hazardous, such drugs as anti-neoplastic drugs used in oncology are particularly dangerous. For example, some anti-neoplastic drugs are considered extremely dangerous, even if contact is made simply upon skin as a liquid or inhaled as a vapor.

To alleviate the likelihood of exposing a hazardous drug to environment outside a drug filled syringe, the presence of a pre-filled flush syringe as part of a kit structure provides a unique opportunity for safety. In this case, the pre-filled syringe and drug dispensing syringe are connected to a common dispensing pathway through a stopcock, where a stopcock is used as a fluid switching component. The stopcock, or any other switching component used according to this invention, should be designed and constructed to permit only one communicating pathway from one of the syringes at a time. Thus, after the syringe is filled with drug in stage 1 (state one of the switching component), the pathway from the drug syringe to the dispensing pathway is closed to the dose syringe and afterward opened to the flush syringe (state two of the switching component). Then, a predetermined amount of flush liquid is dispensed through the dispensing pathway to flush drug from the dispensing pathway and leave flush liquid at the attachment site, as disclosed supra. Similarly in stage 2, after a desired drug volume has been dispensed from the drug syringe (in state one of the switching component), a desired amount of flush liquid is dispensed through the dispensing pathway and through an attached catheter to clear both the associated catheter and connection port (in state two of the switching component) prior to disconnecting the kit parts from the catheter injection port or an associated IV set.

Another advantage of a kit made according to the instant invention is found when administering a short half-life drug (e.g. adenosine). Short half-life drugs, administered through a catheter, must be delivered to their target organ in as short a time as possible. In such cases, it is common practice to connect two syringes to two different “Y” injection sites on an IV set connected to a patient catheter to permit delivery of the short half-life drug from one syringe handled by a first care-giver, followed by delivery of flush from a second syringe by a second care-giver. Having both the drug syringe and flush syringe available to a single dispensing pathway, through a stopcock, provides opportunity for a single care-giver to dispense the short half-life drug, switch the stopcock pathway and immediately dispense the flush syringe. Using the syringe stabilizing clip permits simple motion of a thumb from one syringe plunger stem to the other, while switching the stopcock, to change syringe dispensing modes.

Accordingly, it is a primary object to provide methods and apparatus for preparing and using convenience kits for intravenous medical applications.

It is an object to provide methods and apparatus for preparing and using convenience kits for intravenous delivery of hazardous drugs.

It is an object to provide methods and apparatus for preparing and using convenience kits for intravenous delivery of short half-life drugs.

It is a basic object that parts in a kit transport bag be sterilized while disposed therein.

It is a very important object to provide a kit system which provides access for two syringes.

It is also a very important object to provide an attachment site from a fluid switching component for a drug filling and dispensing syringe as one of the two syringes.

It is yet another very important object to provide an attachment site from a fluid switching component for a pre-filled flush or pre-fillable flush syringe as one of the two syringes.

It is an essential object to provide a method for selectively controlling pathways for fluid flow of the two syringes.

It is a compelling object to provide, for selectively controlling the pathways, a stopcock, affixed to each syringe, which provides a single pathway therefrom.

It is a more compelling object to provide a stopcock which assures fluid within each syringe is kept disparate from fluid within the other syringe.

It is a still more compelling object to provide a fluid switching component having a single input/output pathway, for fluids dispensed from either a dose syringe or a flush syringe, which is geometrically and functionally compatible with general requirements for a needleless connecting port on an IV set or vial access device.

It is a meaningful object to provide a clip for stabilizing the two syringes for single handed operation of the apparatus.

It is another meaningful object to provide a clip which may be used with syringes of various syringe barrel diameters.

It is a critical object to provide a kit for constructing an assembly which significantly reduces makes and breaks required for a predetermined procedure to lessen likelihood of contamination associated with such makes and breaks in a conventionally performed procedure.

It is a crucial object that connectable parts, disposed in the sterilizing pouch, be adjoined to reduce makes and breaks after sterilization.

It is another critical object that such adjoined parts be unreleasibly affixed (unitized) to preclude separation in transport and storage.

It is a another major object that parts be accessible such that the drug syringe can be filled (e.g. in Pharmacy under a laminar flow hood) under conditions which preclude contamination.

It is an object to provide a rigid structure between a syringe used to draw medication from a vial, through a vial access device.

It is an object to provide a sterile cap for closing and protecting the output pathway of the system during transport to a site of use.

It is an object to dispense medication from a kit assembly into a previously primed IV set and therefrom into an IV container preparatory for later delivery to a patient.

It is an object to dispense medication from a kit assembly into a spiked IV set and there from into an IV container for delivery to a patient followed by a flush to clear the medication from dead space associated with a needleless port.

These and other objects and features of the present invention will be apparent from the detailed description taken with reference to accompanying drawings.

### BRIEF DESCRIPTION OF THE DRAWINGS

FIG. 1 is a perspective of a dual syringe assembly, comprising two syringes, a stopcock and a syringe clip, configured according to the instant invention.

FIG. 2A is a schematic drawing of a prior art three-way stopcock having three connecting ports and a rotatable core having a handle which is disposed to show a port closed thereat.

FIG. 2B is a schematic drawing of the three-way stopcock seen in FIG. 2A with the core and handle rotated to close a second port.

FIG. 2B is a schematic drawing of the three-way stopcock seen in FIGS. 2A and 2B with the core and handle rotated to close a third port.

FIG. 3A is a schematic drawing of a two-way stopcock found commonly in commerce and which is similar in structure and position to the three-way stopcock seen in FIG. 2A, but having stops which restrict core rotation (and port closures) to two positions, the first port closure position being seen in FIG. 3A.

FIG. 3B is a schematic drawing of the two-way stopcock seen in FIG. 3A with the core and handle rotated to close a second port.

FIG. 4A is a schematic drawing of a two-way stopcock having a core and handle and associated stops configured according to the present invention and closing the port indicated on the handle.

FIG. 4B is a schematic drawing of the two-way stopcock seen in FIG. 4A, but with the core and handle rotated to close another port.

FIG. 4C is a perspective of a stopcock and an associated short extension set portion of the assembly seen in FIG. 1.

FIG. 4D is a magnified perspective of the stopcock and a portion of the associated extension set seen in FIG. 4C.

FIG. 4E is a front elevation of a PRIOR ART stopcock.

FIG. 4F is a front elevation of a stopcock made in accordance with the present invention.

FIG. 5A is a schematic drawing of another two-way stopcock having a core and handle and associated stops configured according to the present invention and closing the port indicated on the handle.

FIG. 5B is a schematic drawing of the two-way stopcock seen in FIG. 5A, but with the core and handle rotated to close a different port.

FIG. 6A is a schematic drawing of a dual syringe and stopcock assembly with the stopcock core rotated to permit dispensing from a first syringe.

FIG. 6B is a schematic drawing of the dual syringe and stopcock assembly seen in FIG. 6A with the stopcock core rotated to permit dispensing for a second syringe.

FIG. 7A is a front elevation of a dual syringe holder or clip.

FIG. 7B is a front elevation of the dual syringe holder or clip seen in FIG. 7A with an outline of a syringe barrel inserted into a portion of the clip.

FIG. 8 is a front elevation of another syringe holder or clip having a pair of syringe holding cavities, each cavity having a pattern which could hold one of three different barrels of three different syringe sizes.

FIG. 9 is a perspective of a preferred dual syringe holder or clip.

FIG. 10 is a perspective of a dual syringe assembly, comprising two syringes, a stopcock and a syringe clip, configured according to the instant invention, with an oversized syringe barrel disposed in one side of the clip.

FIG. 11 is a perspective of a group of parts assembled for use in a kit according to the present invention.

FIG. 12 is a perspective of the parts, seen in FIG. 11, packaged for sterilization and shipment.

FIG. 13 is a perspective of a dual syringe/stopcock assembly configure according to the present invention with the stopcock disposed for filling a preselected syringe.

FIG. 14 is a perspective of the dual syringe/stopcock assembly seen in FIG. 16, but wherein the predetermined syringe has been filled and stopcock reoriented to permit dispensing of fluid from the other syringe.

FIG. 15 is a perspective of the dual syringe/stopcock assembly seen in FIGS. 13 and 14, but with the stopcock disposed for dispensing fluid from the predetermined syringe.

FIG. 16 is a perspective of the dual syringe/stopcock assembly seen in FIGS. 13-15, but with the stopcock oriented for dispensing fluid from the other syringe.

FIG. 17 is a schematic of a pressure operated fluid switch assembly made in accordance with the present invention.

FIG. 18 is a schematic of a T adapter which is seen in FIG. 17.

FIG. 19 is a schematic of a safety stay of the assembly seen in FIG. 17.

FIG. 20 is a schematic of the T adapter seen in FIG. 17 with a plug displaced from a position seen in FIG. 17.

FIG. 21 is a schematic of the T adapter seen in FIG. 20 with the plug further displaced.

FIG. 22 is a schematic of the T adapter seen in FIGS. 20 and 21 with the plug differently displaced.

FIG. 23 is an exploded view of the assembly seen in FIG. 17.

FIG. 24 is an assembly of the T adapter switch seen in FIG. 17 with a pair of syringes affixed thereto.

FIG. 25 is a PRIOR ART IV set.

FIG. 26 is an IV set having an inverted Y site port affixed and inferiorly disposed relative to a saline containing bag and associated spike of the IV set.

FIG. 27 is a graph of a concentration gradient associated with disposition of a medical syringe being used in a flush mode.

FIG. 28 is a graph of the concentration gradient, seen in FIG. 27, displaced about a connector and a Y site.

FIG. 29 is a graph of a concentration of dispensed medicine about the connector and Y site illustrated in FIG. 28.

FIG. 30 is a graph of a concentration gradient about the connector and Y site seen in FIG. 29 following a saline flush from a pre-filled flush syringe associated with the assembly seen in FIG. 1.

### DETAILED DESCRIPTION OF THE ILLUSTRATED EMBODIMENTS

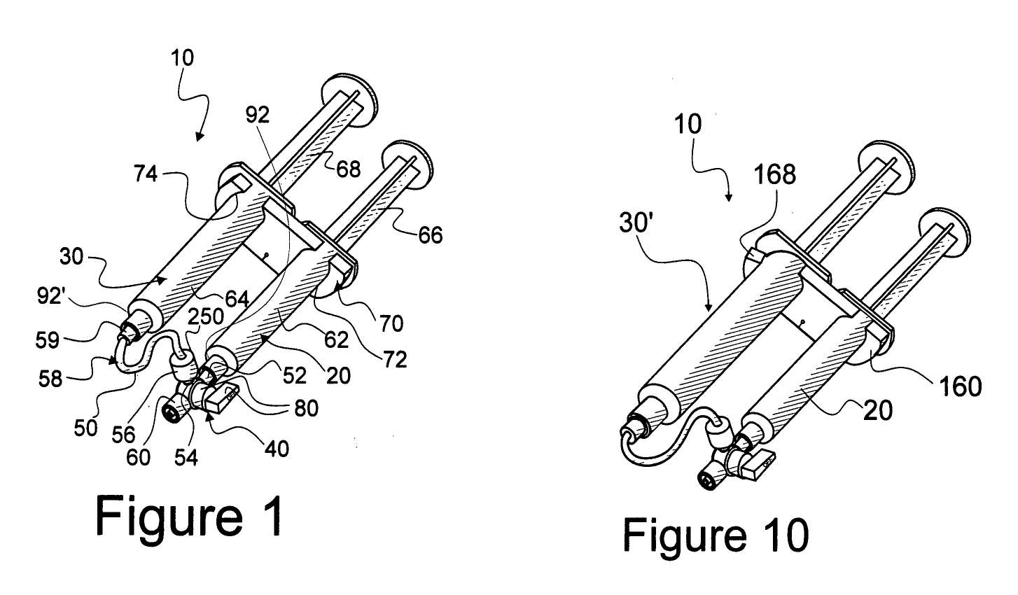

In this description, the term proximal is used to indicate the segment of the device normally closest to the object of the sentence describing its position. The term distal refers to a segment oppositely disposed. Reference is now made to the embodiments illustrated in FIGS. 1-30 wherein like numerals are used to designate like parts throughout. For parts which are similar but not the same as parts originally specified with a given number, a prime of the original numbers is used.

While kits made according to the invention may be configured to provide assemblies for many medical procedures, such as those, for example, involved with injections of Adenosine, antibiotics and drugs for home-care, emergency and pediatrics, disclosure of an exemplary application in the area of hazardous drugs is herein selected to provide details of the instant invention while clearly demonstrating critically important safety and time and work saving features. Reference is now made to FIG. 1 wherein a convenience kit assembly 10 is seen to be readied for dispensing of fluids from a pair of syringes, numbered 20 and 30. Relative to a user, syringes 20 and 30 are interconnected through a stopcock 40 and micro-tubing (short extension) set 50. Each syringe 20 and 30 may be a conventional commercially available medical syringe. One syringe, in particular syringe 30, may be a commercially available pre-filled flush syringe.

Stopcock 40 has three ports, a first port 52 being a female, preferably luer lock, connector which is securely affixed to syringe 20; a second port 54 also being a female, preferably luer lock, connector for connecting to a male connecting port 56 of tubing set 50. At an opposite end, tubing set 50 has a female, preferably luer lock, fitting 59 for secure attachment to syringe 30. Note that port 54 of stopcock 40 is disposed at right angles relative to port 52. Compliance and flexibility of tubing 58 of tubing set 50 permits syringe 30 to be aligned with syringe 20 for purposes disclosed in detail hereafter. A male, preferably luer lock, fitting 60 is exposed for attachment to a port, e.g. an injection port or a vial adapter, wherethrough fluid is communicated.

Further, each syringe, numbers 20 and 30, has a barrel 62 and 64, respectively, and a plunger rod 66 and 68, also respectively. Note that plunger rods 66 and 68 are disposed well outside barrels 62 and 64 indicating both syringes 20 and 30 are filled to a predetermined level (of liquid).

Alignment of syringe 20 to syringe 30 is maintained and assured by a clip 70 having a pair of substantially circular, open slots 72 and 74, Slots 72 and 74 are shaped and formed to provide a releasible support for barrels 62 and 64, respectively. So configured, clip 70 provides a handle or grip whereby first and third fingers of a hand may be disposed outside a perimeter of barrels 62 and 64 with a middle finger of the same hand disposed between the barrels, thereby permitting the thumb of that hand to act upon either plunger rod as desired.

Fluid flow from assembly 10 is controlled by position of rotation of a core and handle 80 of stopcock 40. As seen in FIG. 1, fluid communication into and from syringe 20 is obstructed by the position of core and handle 80. As is explained in detail hereafter, rotation of core and handle 80 to a position obstructing outflow from syringe 30 opens outflow from syringe 20 to controllable permit selective dispensing of fluids from syringes 20 and 30 while keeping fluids within syringes 20 and 30 disparate. It is emphasized that use of a stopcock to control fluid flow is not the only way for such control; however, a stopcock as disclosed herein provides an efficacious way of dealing with requirements for disparate separation of fluids in syringes 20 and 30.

### Stopcocks

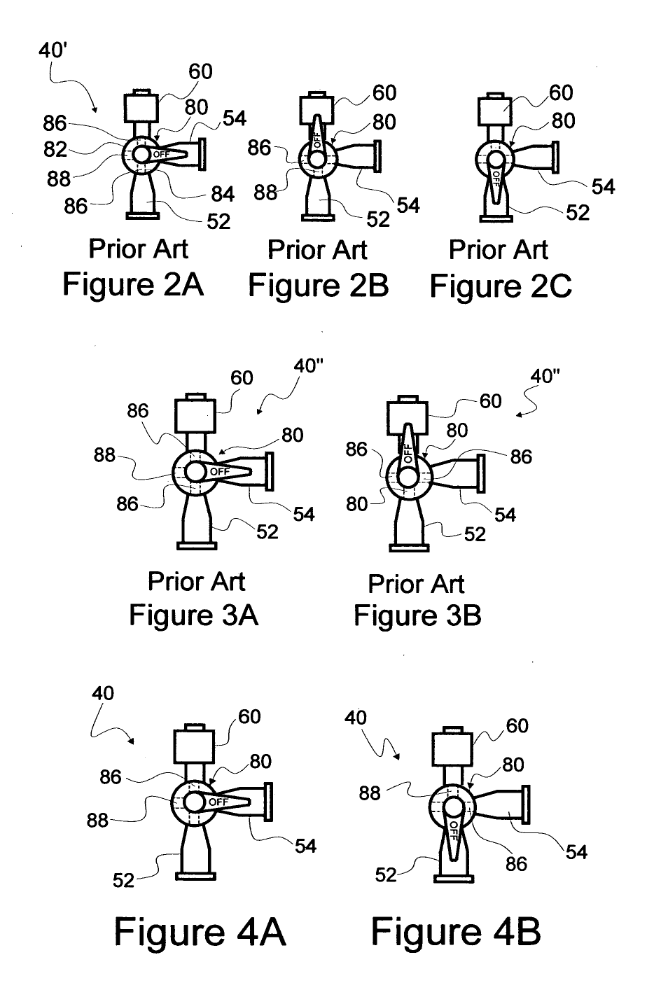

Generally, disposable stopcocks are well known and widely used in medical procedures. A three way stopcock 40′ which is commercially available is seen in FIGS. 2A, 2B and 2C. Stopcock 40′ has three ports 52, 54 and 60 which, except for changes disclosed in detail hereafter, are substantially the same as stopcock 40 (see FIG. 1). As seen in FIG. 2A, within rotatable core 82 of core and handle 80, stopcock 40′ comprises a “T” shaped pathway 84 disposed to obstruct fluid flow through port 54 and permit fluid transmission between ports 52 and 60. Note that pathway 84 can be considered to be comprised of two intersecting pathway segments, individually numbered 86 and 88. Pathway segment 86 is a through hole through core 82, while pathway segment 88 simply intersects pathway segment 86.

Rotating core and handle 80 to a stop associated with port 60, closes port 60 and permits fluid flow between ports 52 and 54 as seen in FIG. 2B. Rotating core and handle 80 to a stop associated with port 52 closes port 52 and permits fluid flow between ports 54 and 60, as seen in FIG. 2C. All of the above steps for stopcock 40′ defines operation of stopcock 40′ to be a three-way stopcock.

A stopcock 40″ seen in FIGS. 3A and 3B is also commonly found in contemporary commerce. Generally ports 52, 54 and 60 of stopcock 40″ are substantially the same as ports 52, 54 and 60 of stopcocks 40 and 40′, except as disclosed in detail hereafter. Note that rotation of core and handle 80 to a stop associated with port 54 as seen in FIG. 3A, closes port 54 and permits fluid flow between ports 52 and 60. However, rotation of core and handle 80 to a stop associated with port 60, closes port 60 and permits fluid flow between ports 52 and 54. In this manner, if port 60 is an output connecting port and ports 52 and 54 are connected to syringes, the syringe connected to port 54 cannot communicate directly with port 60. For this reason, a syringe connected to port 60 is usually affixed thereto to provide dilution fluid to contents of a syringe affixed to port 54. Following such dilution, contents of the syringe affixed to port 52 are dispensed through port 60.

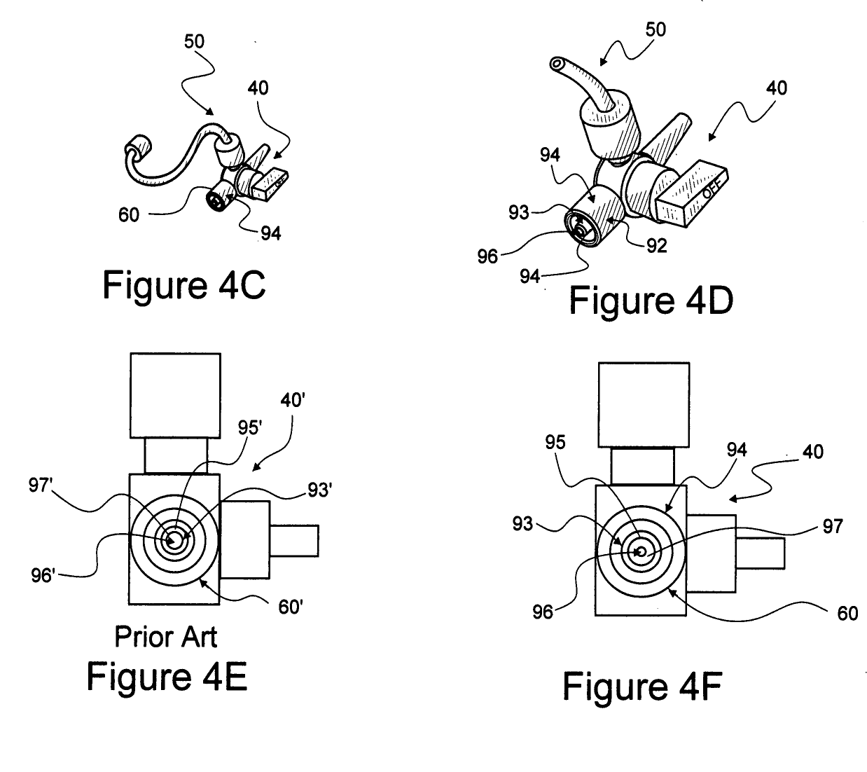

However, in an application where hazardous drugs are to be kept disparate from flushing fluids, it is important that there is no fluid communication between syringes containing such liquids. Therefore, as seen in FIGS. 4A and 4B, core and handle 80 rotation is stopped such that there is no simultaneous communication between ports 52 and 54 along pathway segments 86 and 88. Note, that when core and handle 80 is disposed at a stop associated with port 54, port 54 is closed (switching component state one). When core and handle 80 is disposed at a stop associated with port 52, port 52 is closed (switching component state two). Since stopcock 40 is a two way stopcock, no communication is permitted between ports 54 and 52. In this manner, fluid disposed within port 54 is kept disparate from fluid disposed within port 52.

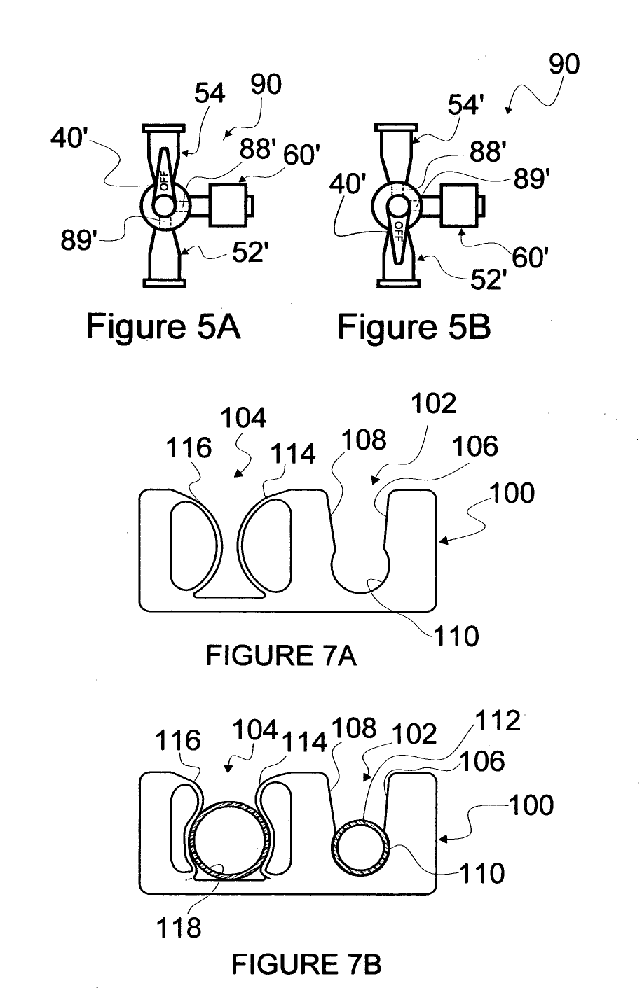

Another stopcock 90 seen in FIGS. 5A and 5B, like stopcock 40, also keeps fluids associated with a pair of syringe ports 52′ and 54′ disparate. Note in FIG. 5A that port 54′ is disposed in line with port 52′. However, a fluid pathway 86′ made up of two connected orthogonally disposed segments 88′ and 89′ permits fluid flow from only one side port 52′ or 54′ to a common output port 60′ at a time. Note in FIG. 5A that pathway 89′ leads from port 52′ to pathway 88′ and output port 60′ where core and handle 80 is at a stop associated with port 54′. Similarly, in FIG. 5B that pathway 88′ leads from port 54′ to pathway 89′ and output port 60′ when core and handle 40′ is at a stop associated with port 52′. While port connections keep fluids of ports 52′ and 54′ disparate in the same manner fluids of ports 52 and 54 are kept disparate in stopcock 40 (see FIGS. 4A and 4B), dead space is decreased in stopcock 40′ (see FIGS. 5A and 5B) relative to dead space in stopcock 40 due to a pathway 89′ which is half the length of pathway 86.

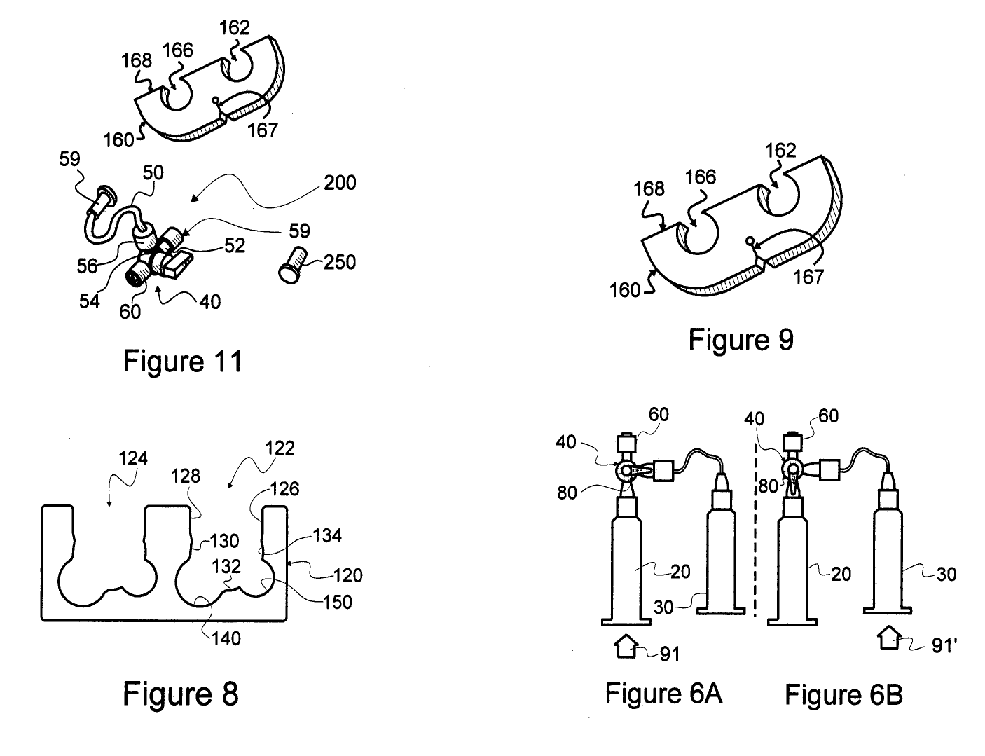

The need for a stopcock such as stopcock 40 (or 90) is exemplified by procedures for use as depicted in FIGS. 6A and 6B. In FIGS. 6A and 6B, arrows replace plunger rods showing direction of displacement of plunger rods. No arrow indicates no plunger rod movement. As a medical procedure associated with the instant invention involves, as a first step, delivering a dose from a syringe dedicated to providing a medication into an injection port. As a second step, immediately dispensing a flush solution into the injection port to flush both the injection port and the catheter itself.

Such is accomplished by simply rotating core and handle 80 to occlude the output pathway of syringe 30, as seen in FIG. 6A, and displacing the plunger rod of syringe 20 in direction of arrow 91. Once desired contents of syringe 20 are dispensed, core and handle 80 of stopcock 40 are displaced to occlude output of syringe 20, as seen in FIG. 6B, and displacement of the plunger rod of syringe 30, in direction of arrow 91′, provides flush solution to the injection port and catheter.

Commonly, needleless fittings are currently used as ports for IV sets affixed to patient catheters and contemporary vial adapters. These fittings have been designed to interface with male syringe luer fittings, such as luer fittings 92 and 92′, affixed to syringes 20 and 30, respectively, see FIG. 1. By standard specifications, male luer fittings have common outside diameters and frustoconical shapes. However, a review of inside diameters of male luer fittings reveals a marked difference between syringe male luer fittings and male luer fittings found on contemporary commercial stopcocks. Exemplary stopcock luer fittings are seen in FIGS. 4C-4F.xxx

A stopcock 40 affixed to a tubing set 50 is seen in FIGS. 4C and 4D. Stopcock 40 and tubing set 50 are magnified in FIG. 4D for clarity of presentation and tubing set 50 is truncated. As seen in FIG. 4D, a proximally disposed luer fitting 60 has a male luer part 93 surrounded by a luer lock 94. As stated supra, luer part 60 has an outside surface 94′ having a standard luer diameter and frustoconical shape, and, to meet requirements of interfacing with needleless connectors, has a medially disposed through bore hole 96.

Stopcock 40 is further magnified in FIG. 4F for additional clarity of presentation. To aid in understanding a basic difference between stopcock 40 and PRIOR ART stopcocks, an example of a PRIOR ART stopcock 40′ is provided in FIG. 4E for comparison. Attention is drawn to male luer part 60 of stopcock 40 in FIG. 4F and a similar male luer part 60′ of stopcock 40′ in FIG. 4E. Note that stopcock 40 has a through bore hole 96 which is relatively smaller than a through bore hole 96′ of stopcock 40′ (see FIG. 4E).

Thus, associated proximal luer face 97, disposed between outside surface 95 and through bore hole 96 is larger in surface area than an outside surface 95′, disposed between outside surface 95′ and through bore hole 96′ of stopcock 40′. Generally, in the past, it is believed that through bore hole 96′ as been defined by draft specifications associated with injection molding. These draft specifications have resulted in the relatively larger size of bore hole 96′. It should be noted that such luer faces are circumferentially defined by outside surfaces having a smallest diameter of approximately 0.150 inches. Such is also true of stopcocks 40 and 40′.

However surface area of syringe luer faces are further defined by a through hole, similar to luer hole 96 of stopcock 40. Diameter of such a syringe through hole is approximately 0.080 inches. Notably, diameter of an exemplary through hole for stopcock 40′ is approximately 0.120 inches. Note that a 0.120 inch diameter through yields a luer face width of about 0.015 inches while a 0.080 inch diameter yields a luer face width of about 0.035 inches. Such a difference in thickness of a luer face is a significant determinant in providing a reliable interface to a needleless connector which has been designed for use with syringe luer dimensions. It is for this reason that stopcock 40 has a significantly smaller through hole diameter than stopcock 40′. Such a decreased size in luer diameter may be achieved by a change in mold design or by affixing a tube having a desired through hole diameter into a larger through hole, such as through bore hole 96′. For purposes of reference, such a stopcock, having a bore hole and luer face thickness similar in dimension and function to a syringe luer connection, is further referenced herein as a needleless compatible connector.

### Clips

Referring once more to FIG. 1, please note that syringe 20 and syringe 30 are aligned, one relative to the other, and held in alignment by clip 70. Clip 70 provides a releasible attachment for each syringe to improve facility of operation of two syringes held in a single hand. Note that a first and third finger may be placed about syringes 20 and 30 while a middle finger may be placed between the two syringes in such a manner that the thumb of the hand can be used to displace each syringe rod, 66 and 68. It is important that clip 70 holds each syringe securely, but releasibly, such that either syringe may be removed from clip 70 for purposes which require a separated syringe, such as placing a syringe in a syringe pump.

Clips for assembly 10 may be made in many forms within the scope of the instant invention. Basic criteria for such clips are that the clip must provide sufficient stability for assembly 10 that two syringes may be facilely employed in a single hand and the syringe attachment must be secure, but releasible. Another optional requirement is that the clip be usable for a predetermined range of syringe barrel sizes.

A syringe clip 100, made according to the instant invention, is seen in FIGS. 7A and 7B. Syringe clip 100 has a pair of slots 102 and 104 into which syringes may be displaced. As seen in FIG. 7A slot 102 comprises a pair of sides 106 and 108 which converge toward an open circular slot 110 which is sized and shaped to conform to a single predetermined syringe barrel size. Note, in FIG. 7B that a syringe barrel 112 (seen in cross section), is disposed in slot 102.

However, it is preferred that a clip be useful for more than one syringe barrel size. For this reason, slot 104 comprises a pair of compliant ribs 114 and 116 which forgivingly separate when a syringe barrel is displaced there into (see a cross section of a syringe barrel 118 disposed in slot 104. Ribs 114 and 116 must exert sufficient force against barrel 118 to retain barrel 118 in slot 104 once so disposed.

Clip 100 should be sufficiently thick to hold each inserted syringe barrel in position throughout a predetermined medical procedure associated with assembly 10. Clips like clip 100 may be injection molded using polypropylene.

A clip which is specifically designed to hold syringe barrels of a variety of sizes is seen in FIG. 8. As seen in FIG. 8, a clip 120 comprises two identical slots 122 and 124. As slots 122 and 124 are identical, only characteristics of slot 122 will be disclosed in detail. Slot 122 has a pair of converging sides 126 and 128 and a pattern which is sized and shaped to grasp a large syringe barrel (not shown) within edges 130, 132 and 134. Offset from slot 122 is a smaller circular slot 140 which is sized and shaped to grasp a smaller syringe barrel (also not shown). On an opposite side of slot 122 is yet another still smaller circular slot 150 sized and shaped to grasp a still smaller syringe (also not shown). In this manner, a single clip 120 may be used to hold one of three different sized syringe barrels within each slot 122 and 124. Similar to clip 100, clip 120 may be injection molded from polypropylene.

A preferred clip 160 is seen in FIG. 9. Clip 160 comprises a pair of circular slots, numbered 162 and 166, which open superiorly to permit insertion (and retrieval) of a syringe barrel. The circular slots each have a diameter which is smaller than the smallest syringe barrel used in assembly 10. Further, clip 160 has a centrally disposed slit and hole 167 sized and shaped to permit clip 160 (and assembly 10) to be facilely suspended from tubing available at the site of use.

Clip 160 is preferably made of a substantially rigid closed cell foam material. As such clip 160 may be made by stamping out of a sheet of material. While clip 160 may be made in various thicknesses (e.g. from 0.25 to 0.50 inches), a thickness of 0.375 inches is preferred to reduce likelihood of inadvertently concealing indicia generally placed on the barrel of a syringe. Closed cell foam, from which clip 160 is made, is particularly compatible for use as a barrel holder for assembly 10. The foam permits a tight grasp of an inserted barrel which is just larger than the diameter of the slot and yields when a much larger barrel is inserted to provide a stabilizing clasp upon the larger syringe barrel.

An example of the manner in which clip 160 yields to a larger syringe is seen in FIG. 10 wherein an assembly 10 comprises a syringe 30′ which is substantially larger in diameter than syringe 30 seen in FIG. 1. Note in FIG. 10 that an outside arm 168 is displaced from an original position as seen in FIG. 9. While insertion of larger syringe 30′ causes displacement of arm 168, clip 160 still acts as an adequate stabilizing clasp about syringe 30′.

### Kit Packaging

Generally, kit components, to be sterilized, are displaced into a pouch, sealed there within and sterilized by a predetermined method of sterilization (such as gamma radiation, ethylene oxide, etc.). One of the primary objects of the instant invention is to decrease numbers of makes and breaks after sterilization to as few as possible. For this purpose, as much as possible, kit parts which are joined for use in assembly 10 are securely affixed one to another prior to being sterilized. It is important that these parts remain affixed one to another through all phases of kit use.

For this reason, it is recommended that these parts be unitized parts, becoming even as a single unitized part 200 (i.e. be adhesively interconnected where possible), as seen in FIG. 11 Where such is not possible the parts should be tightly mechanically secured. In part 200, port 54 of stopcock 40 is affixed to a male fitting 56 of a short extension set 50. A female fitting 59 and a male fitting 60 are left open for purposes which are disclosed in detail hereafter. As a cap 250 may be later used as a sterility protecting cover after a syringe 20 filling procedure, cap 250 is included with the other sterilized parts. Also included, for convenience, is a clip 160.

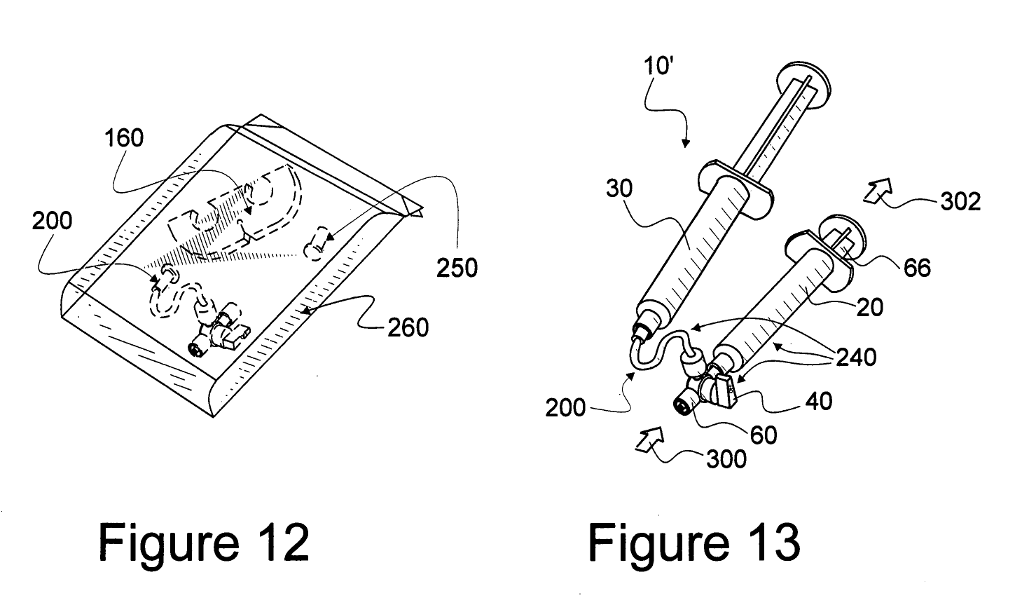

A preferred mode of packaging kit parts including unitized part 200, cap 250 and clip 160 is seen in FIGS. 12. Note that the kit parts are disposed and sealed within a sterilizable peel pouch or wrap 260 wherein contained parts are processed by gamma radiation, ethylene oxide or other sterilization process.

Note that drug syringe 20 and flush syringe 30 are not included in items sterilized in peel pouch or wrap 260. Generally, both syringes are readily available at a using institution and a kit having a particular syringe may not match syringes selected for use by a that institution.

### Alternative to Stopcock

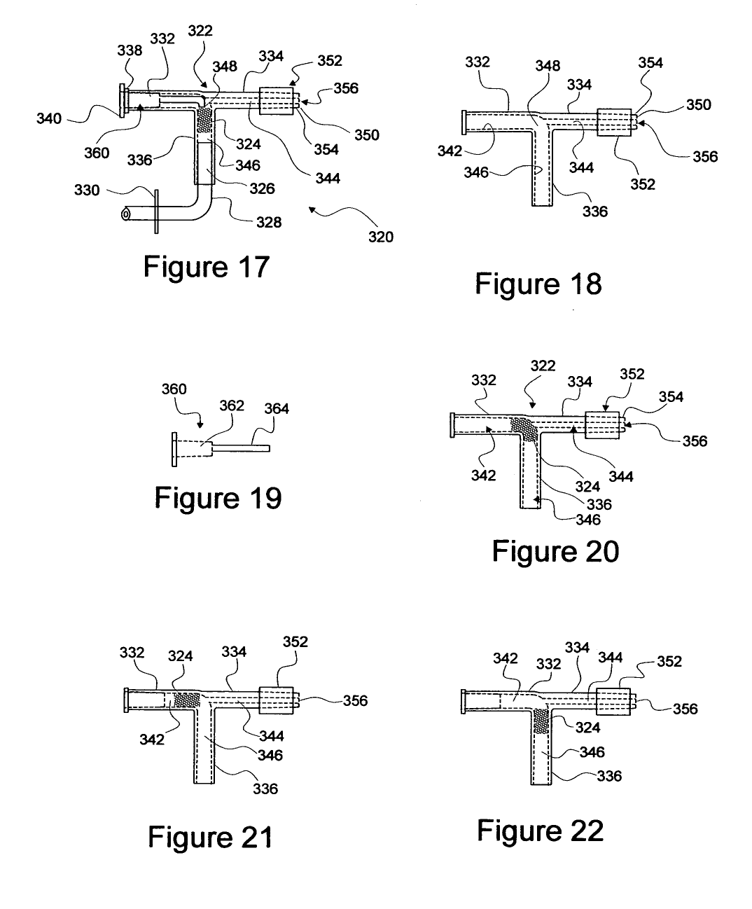

While use of a stopcock, such as stopcock 40, is in accord with the present invention, an alternative, which requires no external manual switching is provided by a pressure actuated fluid switching apparatus 320, seen in FIGS. 17-24. As seen in FIG. 17, apparatus 320 comprises a T adapter 322, a displaceable plug 324, a tube 326 of an associated extension set 328, and a slide clamp 330. T adapter 322 further has three definitive legs, numbered 332, 334 and 336. Note that leg 336 is orthogonally disposed relative to legs 332 and 334 to form a “T” shape. Leg 332 has an externally disposed female luer connector 338, having a luer lock flange 340 and a luer fitting 338 internal to leg 332.

As is common with T adaptors, each leg 332, 334 and 336 comprises a through fluid pathway 342, 344 and 346, respectively as delimited by dashed lines, as may be seen in FIG. 18. All pathways 342, 344 and 346 converge to a central cavity 348. At an exterior terminating end 350 of leg 344 is a luer lock connector 352. Connector 352 comprises a luer fitting 354 having a centrally disposed exterior orifice 356 for pathway 344. It is important to note that orifice 356 and end 350 have structure and geometry consistent with a conventional syringe such that luer lock connector 352 may be used for an interface with a needleless connector.

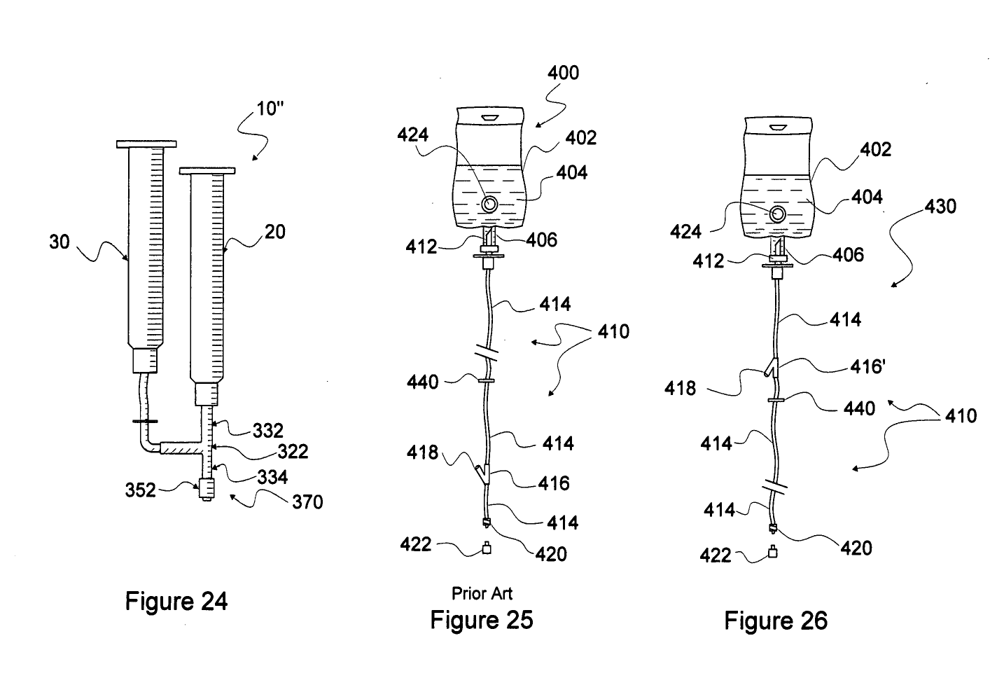

In addition to T adapter 320, a safety stay 360 is seen disposed in pathway 342 of leg 332. Safety stay 360 is disposed within pathway 342 until a syringe is affixed to female luer connector 338. Note, as seen in FIG. 19 that safety stay 360, as well as having a male luer geometric section 362, has an elongated section 364 which follows pathway 342 to provide a stop for plug 324. In this manner, plug 324 is retained in pathway 346 and pathways 342 and 344 are thereby unobstructed by plug 324, permitting free pathways 342 and 344 (switching component state one) to be used when filling a syringe initially connected to luer connector 338 (see FIG. 17). Thus, removal of safety stay 360, and attachment of a medicinal syringe 20 to connector 338, is an important step in constructing an assembly 10″, as seen in FIG. 24. Also a pre-filled flush syringe may be affixed as seen in FIG. 24 to complete assembly 10″.

Note, that so constructed, syringe 20 in combination with legs 332 and 334 of T adapter 322 and luer lock connector 352 form a linear rigid member 370 which may be affixed to a needleless port of either a catheter or a vial adapter for fluid transfer. Syringe 30 is affixed to leg 336 within pathway 346 where through flush solution is delivered.

Determining an open flow pathway through which fluid may be transferred relative to syringe 20 or syringe 30 is entirely dependent upon pressure exerted within a syringe 20 or a syringe 30. Note, in an initial state, as depicted in FIG. 17, plug 324 is disposed to occlude pathway 346, leaving pathways 342 and 344 open (switching component state one). In this initial state, safety stay 360 is removed and a syringe 20 is affixed to connector 338. For filling, for example from a vial, a vial adapter is affixed to luer lock connector 352 and a vial of fluid is thereby accessed. Syringe 20 is then filled with a desired volume of medication.

Once syringe 20 is properly filled (and primed), pressure is applied to a pre-filled syringe 30 affixed to extension set 328 (see in FIG. 24). Application of pressure in an attached syringe 30 displaces plug 324 from a site seen in FIG. 17 past a site seen in FIG. 20. Note that, as plug 324 passes through the site seen in FIG. 20, both pathways 346 and 342 are occluded (closed). Further application of pressure from syringe 30 displaces plug 324 to a site seen in FIG. 21 whereat plug 324 occludes pathway 342 and pathway 346 is open for delivery of fluid from syringe 30 into pathway 344 (switching component state two). If, for example, pressure is being applied to syringe 30 as part of a syringe 20 filling stage, pathways 344 and 346 are joined for priming and flushing of parts associated with connector 352 and orifice 356. Once flushing is complete, assembly 10″ may be removed from the needleless connector and a tip cap may be affixed to connector 352 for transport of assembly 10″ to a site of use.

At the site of use, the tip cap is removed and connector 352 is connected to a medical dispensing site, such as to a needleless connector of a catheter IV set. Once so affixed, medication may be delivered. To accomplish such delivery, pressure is applied to syringe 20 to displace plug 324 from a site as seen in FIG. 21 to a pathway 346 occluding site as seen in FIG. 21, opening pathway 342 to pathway 344. Once a desired volume of medication is dispensed, pressure is applied to syringe 30 to once again open pathway 346 to pathway 344 so that parts associated with connector 352 and orifice 356 may be flushed with liquid from syringe 30. Once so flushed, connector 352 may be disconnected and assembly 10″ (see FIG. 24) disposed of according to institutional protocol.

An exploded view of parts related to T adapter 322 is seen in FIG. 23. Parts are assembled along dashed lines, generally numbered 380. Safety stay 360 may be injection molded from a pliant synthetic resinous material such as polypropylene. T adapters are generally available in commerce and T adapter 322 may be made from material similar to material used for generally available T adapters. IV set 328 utilizes tubing which is commonly used in commercially available IV sets. Also, slide clamp 330 may be selected from a wide variety of slide clamps currently available in commerce. A female luer connector 390 is affixed to complete IV set 328. Plug 324 should be made from a pliant and resilient synthetic resinous material having a “tough” surface against abrasions due to inherent parting lines due to T adapter 322 injection molding. Such a material may be a compliant, flexible synthetic resinous material, such as silicone or butyl rubber. A particularly applicable candidate material which may be used is a medical grade SANTOPRENE® TPZ 18-55, by EXXON Mobil.

### Methods of Preparation and Use

Reference is now made to FIG. 13 wherein an assembly 10′, which is assembly 10 without clip 70, is seen. Note that assembly 10′ (a part of assembly 10) is constructed by attaching an empty drug injection syringe 20 and a pre-filled flush syringe 30 to a unitized part 200 (see FIG. 13) to make a completed assembly. To reduce likelihood of contamination, such attachments should be performed in a clean, controlled environment, such as within a safe area of a laminar flow hood.

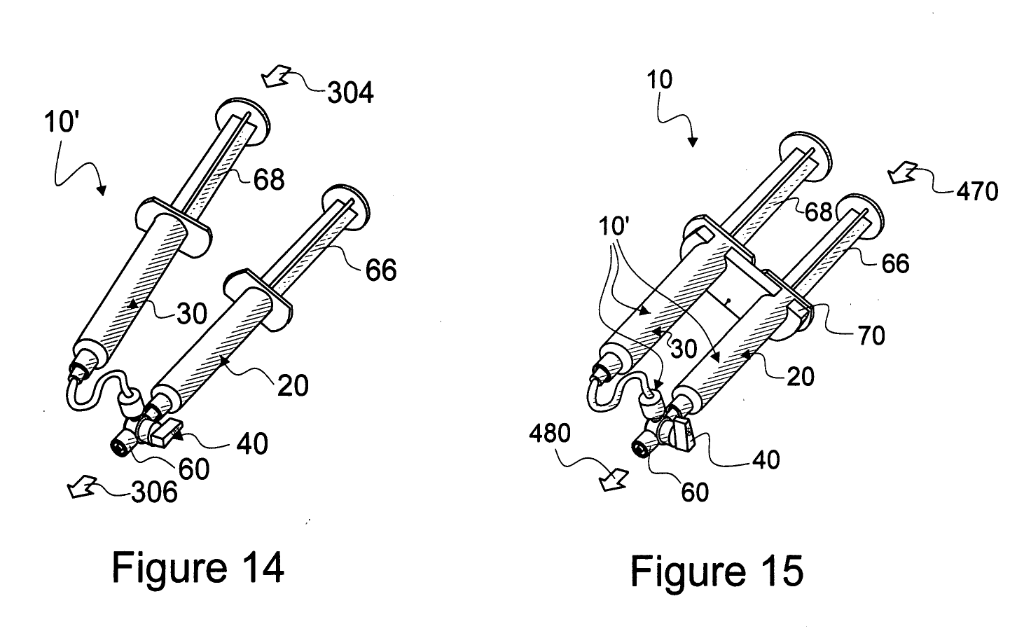

With stopcock 40 disposed for filling syringe 20, as seen in FIG. 13, male fitting 60 of stopcock 40 is affixed to a source of drug (e.g. a vial adapter having a needleless connector) to be transferred to syringe 20 according to institutional protocol. As is well understood by clinicians trained in use of syringes, medication or drug is drawn into syringe 20 in direction of arrow 300 by retracting plunger rod 66 in direction of arrow 302. Once syringe 20 is filled, and primed, a predetermined amount of flush solution is dispensed from pre-filled flush syringe 30 as seen in FIG. 14. To accomplish this, stopcock 40 is disposed to permit fluid flow from syringe 30 through fitting 60. Plunger rod 68 is displaced in direction of arrow 304 to dispense flush solution outward from fitting 60 in direction of arrow 306. It is recommended that, for 10 ml flush syringes, one to two milliliters of flush solution be dispensed through flush fitting 60, though volumes may vary dependent upon character of drug in syringe 20. Note that by flushing fitting 60 hazardous drug resident at fitting 60 is displaced by flush solution.

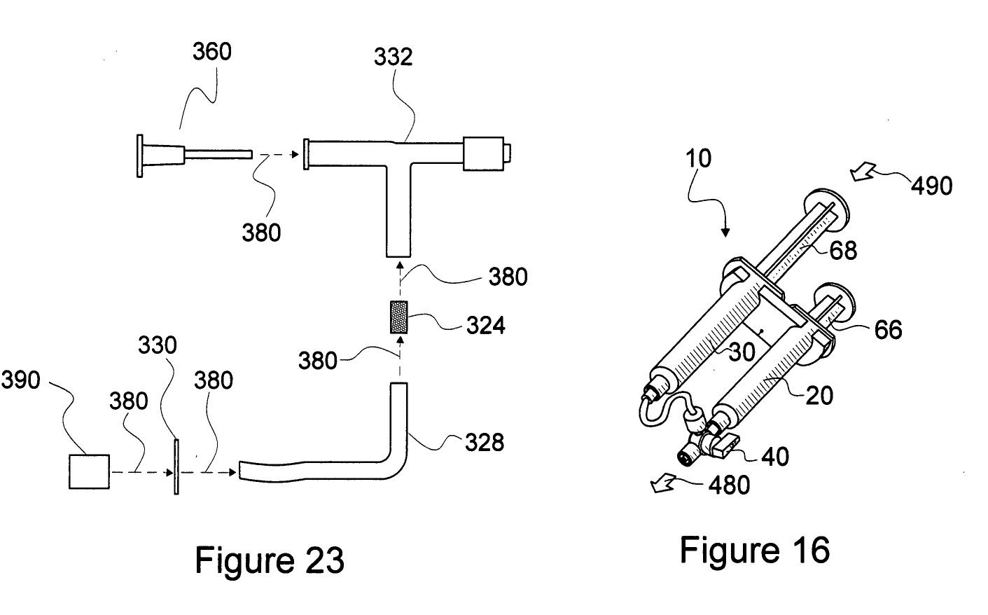

Once syringe 20 is filled and fitting 60 is flushed, stopcock 40 should remain in the open flush pathway state. Fitting 60 should be capped (preferably with provided cap 250 (see FIG. 11). At any desired time, clip 70 may be affixed thereto (as seen in FIGS. 1 and 16) to form assembly 10. Then, following institutional protocol, a prepared system 10 is delivered to a site of use.

A site where a drug is dispensed from syringe 20 may be varied. Examples of such sites are provided hereafter:

### Pharmacy Dispensing

A first exemplary site is in pharmacy, likely where system 10 has been prepared. In such a case, medication may commonly be dispensed into a saline bag through some kind of bag injector site. A pathway for injecting might include a secondary spike injection site, a side injection port or a pathway through an associated catheter.

An exemplary PRIOR ART IV solution bag/IV set combination 400 is seen in FIG. 25. Combination 400 generally comprises an IV bag 402 filled with IV solution 404, a spike insertion port 406 and an injection set 410 having a superiorly disposed spike 412 (introduced into port 406), an elongated length of medical tubing (generally numbered 414), an inferiorly disposed Y injection site 416 (usually fitted with a needleless connector 418) and a needleless connector compatible fitting 420 for being ultimately affixed to a patient catheter. A tip cap 422 is also provided to close IV set 410 for transport.

Note that Y site 416 is disposed for inferiorly directed injection into tubing 414, likely at a patient site. Note also that IV bag 402 has an injection port 424 where through a medication may be dispensed by needle insertion. As ports, like port 424, may leak, such are not considered by inventors to be appropriate interfaces for hazardous drugs.

To provide a safer interface for dispensing hazardous drugs into an IV bag, such as bag 402, an IV solution bag/IV set combination 430, made according to the present invention is seen in FIG. 26. Combination 430, as an example, may comprise an IV bag 402 filled with IV solution 404, a spike insertion port 406 and an injection set 410 having a superiorly disposed spike 412 (introduced into port 406), an elongated length of medical tubing (generally numbered 414), a Y injection site 416′ (superiorly disposed relative to Y site 416 and fitted with a needleless connector 418) and a needleless connector compatible fitting 420 for being ultimately affixed to a patient catheter. A tip cap 422 is also provided to close IV set 410 for transport.

Note that Y site 416′ is disposed for superiorly directed injection into tubing. Note also that IV bag 402 an injection port 424 is not needed as Y injection site 416′ may be safely and efficaciously used for dispensing medication into solution 404. Note: Before dispensing medication into bag 402, assembly 410 should be primed with solution from the IV container. Then, with assembly 10 (see FIG. 1), or 10″ (see FIG. 24), affixed to Y site 416′ through needleless connector 418 a medication/flush cycle may begin. A slide clamp 440 inferiorly disposed, relative to Y site 416′, is oriented about tubing 414 to occlude tubing 414.

In the case of assembly 10, stopcock 40 is adjusted to provide a pathway from syringe 20 through luer connector 60 and there through Y site 416′ and into bag 402 and solution 404. Once a desired volume of medication is so dispensed, stopcock 40 is adjusted to provide a pathway from syringe 30 through luer connector 60 and needleless connector 418 and into bag 402 to permit flushing of luer connector 60, needleless connector 418, Y site 416′, tubing 414 superior to Y site 416′ and associated spike 412. Once flushing is completed, assembly 10 may be removed with safety and slide clamp 440 adjusted to permit flow through tubing 414. For safety, assembly 10 should be disposed of following institutional protocol.

### At Patient Dispensing

Note that, when, for example, assembly 10 is displaced for use at a medication delivery site, a clinician may perform the dispensing operation single handed, dispensing at will from either of the two syringes, 20 and 30. For catheter related dispensing, fitting 60 is securely, but releasibly affixed to a receiving catheter or other receptacle fitting (at least for hazardous drugs, the fitting should be a needleless connector). Stopcock 40 is set to provide an open pathway from syringe 20 to fitting 60. Plunger rod 66 is displaced in direction of arrow 470 to dispense medicant through fitting 60 in direction of arrow 480 for its designated purpose, as seen in FIG. 16. Note that by grasping assembly 10 about syringes 20 and 30 with the index and third fingers and placing the middle finger of a hand between syringes 20 and 30, the thumb of the hand can facilely displace plunger rods 66 and 68.

Once a desired amount of fluid of syringe 20 is displaced therefrom, stopcock 40 is displaced to obstruct flow of fluid from syringe 20 and open the fluid flow pathway from syringe 30. Generally, sufficient fluid is dispensed from syringe 30 by displacing plunger rod 66 in direction of arrow 490 to flush fitting 60, an associated IV connector and a catheter or other communicating fluid line, as seen in FIG. 16. For a single use application of assembly 10, plunger rods 66 and 68 are fully displaced, spent assembly 10 is delivered to a disposable receptacle according to institutional protocol.

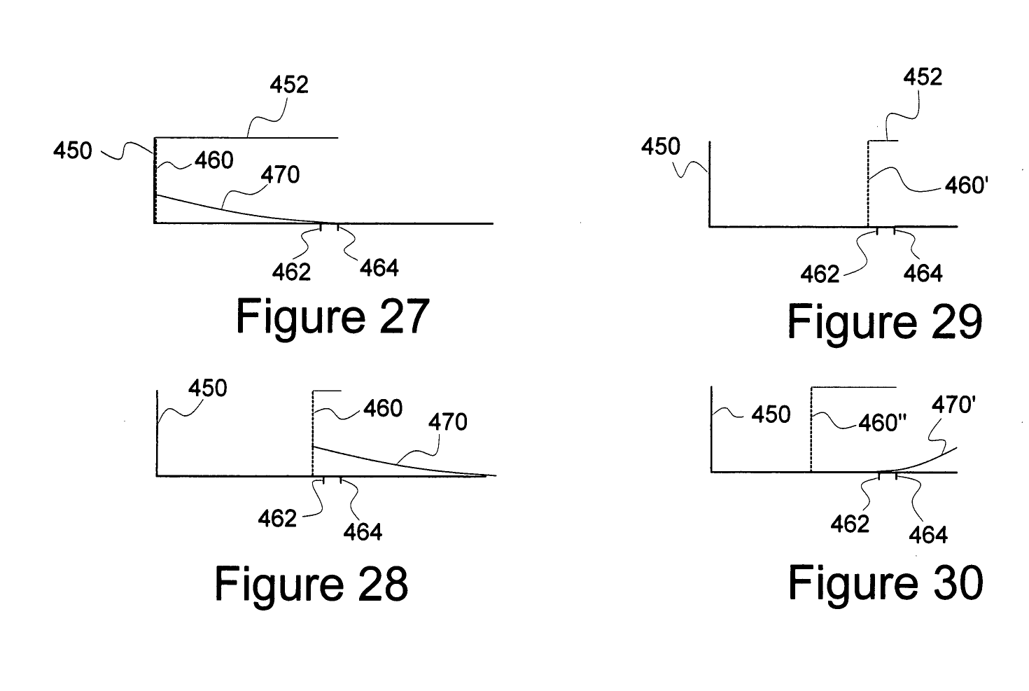

In some institutions, it is a practice to attempt to flush a catheter connector (usually needleless) by drawing flush, into the syringe from which medication was dispensed, from a saline drip line following medication delivery. Applicants feel a necessity to stipulate a concern relative to such a practice. As an example, such a practice may yield a distribution of medication following such flushing as indicated in FIGS. 27 and 28.

In graphs of FIGS. 27-30, the “Y” or vertical axis 450 of each graph represents a measure of drug concentration. The “X” or horizontal axis of each graph a measure of relative distance between points enumerated on the “X” axis (effectively plunger rod displacement). A relative disposition of an exposed face of a syringe plunger which is used to displace fluid from a syringe is designated by vertical line 460. A small vertical line 462 designates position of a needleless connector interface and a second small vertical line 464 designates position of a point where an attached section of a “Y” connector communicates with an associated IV set. A downwardly sloping line 470 indicates a concentration gradient away from a syringe plunger tip face 460.

As seen in FIG. 27, an original medication concentration 452 is contained in and delivered from, in the more general case, a medication syringe. An attempt to draw in saline solution from a saline drip line for a flushing purpose results in some mixing and a concentration gradient which is plotted for example as gradient 470 as seen in FIG. 27. Note that concentration of gradient 470 is highest at plunger face 460 where actual concentration is dependent upon mixing between original medication resident in dead space (including the attached section of the “Y” connector. When plunger face 460 is displaced to flush past points 462 and 464, concentration gradient 470 is effectively displaced to provide the highest level of concentration in the region of points 462 and 464. Because point 462 represents a break point when the medication syringe is detached from the “Y” site, some concern is believed to be in order.

On another hand, if assemblies 10 or 10″ are used for flushing, with a pre-filled flush syringe providing flush solution external to a “Y” site, concentration at the end of dispensing yields an original concentration 452 at a driving plunger face (e.g. face 460′ in FIG. 29). Interestingly, when a stopcock is switched, such as stopcock 40 of assembly 10, medicine concentration within a pre-flush syringe is inherently free of medication (having a zero medication concentration). Therefore, as a driving plunger face (e.g. face 460″ of a pre-filled syringe 30) is displaced to dispense flush from syringe 30, concentration gradient is generally of the form of curve 470′ of FIG. 30. For this reason, it is intuitive that flushing is more efficacious when using a flush syringe than when using a medicine delivery syringe to draw and redispense a saline/medicine mix in an attempt to clear a needleless connector and associated “Y” site.

What is claimed and desired to be secured by Letters Patent is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A medical convenience kit for assembling an apparatus for measuring, filling and dispensing medication and flush solutions through connections to IV sets and patient lines and catheters while improving safety and efficacy of such procedures by requiring fewer post-sterilization makes and breaks than like procedures performed with conventional components, by facilitating dispensing of flush solutions, by providing for flushing of IV set and patient line and catheter connecting fittings before breaking such connections, by providing a two syringe assembly which provides for selectively dispensing from either of the two syringes while obstructing fluid displacement from the other syringe, said kit comprising:
a fluid switching apparatus comprising a first fitting for connecting to a medication delivery syringe, a second fitting for connecting to a pre-filled flush syringe, and a third fitting for accessing a medication container to thereby communicate a predetermined volume of a medication into the medication delivery syringe and, alternatively, for connecting to an external port for the purpose of dispensing fluid from the apparatus;
said fluid switching apparatus further comprising three intersecting pathways and an associated displaceable switching component disposed for selectively obstructing and permitting fluid flow through each of said intersecting pathways, a first pathway being disposed between said first fitting and said displaceable switching component, said second pathway being disposed between said second fitting and said displaceable switching component and said third pathway being disposed between said third fitting and said displaceable switching component;
said displaceable switching component comprising structure which, when displaced to a first state, selectively provides a fluid communication pathway only from said first pathway to said third pathway and when displaced to a second selected state provides a fluid communication pathway only between said second pathway and said third pathway, but under no conditions, in any state, provides a fluid communication pathway between said first and second pathways; and
said third fitting comprising an assembly which provides the pathway through which fluid is externally communicated and further comprising an associated connecting geometry having needleless connector compatible construction.

22. The medical convenience kit according to claim 21 further comprising a connecting link affixed to said second fitting whereby an attached flush syringe is aligned with the medication delivery syringe such that both syringes may be grasped and fluid be manually delivered from either syringe by a single hand.

23. The medical convenience kit according to claim 21 wherein said third fitting comprises a male luer lock fitting.

24. The medical convenience kit according to claim 21 wherein said fluid switching apparatus comprises in-line structure disposed between said first fitting and said third fitting.

25. The medical convenience kit according to claim 21 wherein said connecting link is a short extension set, connecting on one end to said second fitting and on the other end to a flush syringe.

26. The medical convenience kit according to claim 21 wherein said fluid switching apparatus is a stopcock.

27. The medical convenience kit according to claim 26 wherein said stopcock is a two-way stopcock having a core which is displaceable to two fluid conducting states, a first state only interconnecting said first pathway with said third pathway and said second state only interconnecting said second pathway with said third pathway.

28. The medical convenience kit according to claim 21 wherein said fluid switching apparatus is a pressure actuated switch comprising an obstructing plug which is displaced to a first state to open a pathway via a pressure generated within a medical delivery syringe, affixed to the first fitting, and communicated to the first pathway to provide a communicating intermediate pathway from the first pathway to the third pathway thereby and to a second state to open a pathway via a pressure generated within a flush syringe, affixed to the second fitting, and communicated to the second pathway to provide a communicating intermediate pathway from the second pathway to the third pathway thereby.

29. The medical convenience kit according to claim 22 further comprising a clip, which is releasibly affixed to a medication delivery syringe and a flush syringe to thereby stabilize and hold both syringes in alignment for facile management by a single hand.

30. A method for measuring, filling and dispensing medication and flush solutions through connections to IV sets and patient lines and catheters while improving safety and efficacy of such procedures by requiring fewer post-sterilization makes and breaks than like procedures performed with conventional components, by facilitating dispensing of flush solutions, by providing for flushing of IV set and patient line and catheter connecting fittings before breaking such connections, by providing a two syringe assembly for selectively dispensing from either of the two syringes, said method comprising the following steps:
(a) providing a packaged, sterilized kit comprising:
a fluid switching apparatus comprising a first fitting for connecting to a medication delivery syringe, a second fitting through which connection is made to a dedicated flush syringe, and a third fitting for accessing a medication container to thereby communicate a predetermined volume of a medication into the medication delivery syringe and, alternatively, for connecting to an external port for the purpose of dispensing fluid from the apparatus;
said fluid switching apparatus further comprising three intersecting pathways and an associated displaceable switching component disposed for selectively obstructing and permitting fluid flow through each of said intersecting pathways, a first pathway being disposed between said first fitting and said displaceable switching component, said second pathway being disposed between said second fitting and said displaceable switching component and said third pathway being disposed between said third fitting and said displaceable switching component;
said displaceable switching component comprising structure which, when displaced to a first state, selectively provides a fluid communication pathway only from said first pathway to said third pathway and when displaced to a second selected state provides a fluid communication pathway only between said second pathway and said third pathway; and
said third fitting comprising an assembly which provides a pathway through which fluid is externally communicated and further comprising an associated connecting geometry having needleless connector compatible construction.
(b) disposing said kit in a work area where assembled parts are not contaminated and, then, opening said kit for access to provided parts;
(c) displacing said switching component to provide a communicating pathway between said first and third pathways and affixing a medical delivery syringe to said first fitting;
(d) affixing a pre-filled flush syringe to said second fitting to provide a complete assembly;
(e) affixing a vial access device having a needleless connector to said third fitting;
(f) accessing a medication disposed in a predetermined vial through the vial access device;
(g) drawing a predetermined volume of medication into the medication syringe;
(h) displacing the switching component to provide a communicating pathway between said second and third pathways;
(i) dispensing a predetermined volume of flush solution through said third fitting and needleless connector affixed thereto; and
(j) disconnecting said third fitting from said needleless connector to provide a free assembly thereby.

31. A method according to claim 30 comprising the following additional steps of:
(k) providing a luer tip cap and releasibly affixing said tip cap to said third fitting following step (j) to provide a transportable assembly and
(l) transporting the transportable assembly to a site of use.

32. The method according to claim 31 comprising the next steps of:
(m) removing said tip cap from the transportable assembly and
(n) affixing said second fitting to a needleless IV port connector affixed to a patient line or catheter as a first make at the site of use.

33. The method according to claim 32 comprising the next steps of:
(o) displacing said switching component to the first state; and
(p) dispensing a predetermined volume of medication from the medication delivery syringe through the needleless connector.

34. The method according to claim 33 comprising the next steps of:
(q) displacing said switching component to the second state and dispensing a predetermined fluid volume from the flush syringe through the needleless connector.
(r) detaching the third fitting from the needleless connector as a single break at the site of use; and
(s) disposing of the residual transportable assembly according to institutional protocol.

35. A method according to claim 30 comprising the steps of:
(t) providing an IV solution bag/IV set combination comprising a predetermined volume of IV solution in the bag and a needleless connector for fluid transfer;
(u) pre-priming the IV set prior to injection of medication; and
(v) affixing said third fitting to the needeless connector of the combination.

36. A method according to claim 35 comprising the steps of:
(w) displacing said switching component to the first state; and
(x) dispensing a predetermined volume of medication through the needleless connector of the combination.

37. A method according to claim 36 comprising the steps of:
(y) displacing said switching component to the second state; and
(z) dispensing a predetermined volume of flush solution through the needleless connector of the combination.

38. The method according to claim 37 comprising the steps of:
(aa) detaching the second fitting from the needleless connector; and
(bb) disposing of the residual complete assembly according to institutional protocol.

39. The method according to claim 30 comprising steps of:
(cc) further providing a connecting link affixed to said second fitting whereby an attached flush syringe is aligned with the medication delivery syringe whereby both syringes may be grasped and operated by a single hand to manually deliver fluid from either syringe thereby.

40. The method according to claim 39 comprising the steps of:
(dd) providing a clip; and
(ee) affixing the medical syringe and flush syringe to the clip following completion of steps (c) and (d), thereby facilitating holding both syringes and actuating either syringe with a single hand.
